(19) United States
(12) Patent Application Publication
(10) Pub. No.: US 2007/0270710 A1
(43) Pub. Date: Nov. 22, 2007

(54) Device Used for Needle Biopsy

(76) Inventors: Michael Frass, Modling (AT); Amir Kurtaran, Purkersdorf (AT); Klaus Kaserer, Wien (AT); Leopold Halling, Wien (AT)

Correspondence Address: RATNERPRESTIA, P O BOX 980, VALLEY FORGE, PA, 19482-0980, US

(21) Appl. No.: 10/567,498

(22) PCT Filed: Aug. 9, 2004
(86) PCT No.: PCT/AT04/00282

§ 371 Date: Mar. 2, 2007

(30) Foreign Application Priority Data
Aug. 7, 2003 (AT) A 1252/2003
Sep. 11, 2003 (AT) A 1440/2003
Apr. 27, 2004 (AT) A 722/2004

(51) Int. Cl. A61B 10/02

(52) U.S. Cl. 600/567

(57) ABSTRACT

A device for needle biopsy with a syringe cylinder, with a plunger displaceable therein as well as with a needle means, which has a plurality of puncture needles and whose channels open into the interior of the cylinder, wherein a ventilation means, by which the volume between the bottom and the plunger can be temporarily connected with the environment, is provided for the interior of the syringe cylinder.

The present invention pertains to a device for needle biopsy with a syringe cylinder, with a plunger displaceable therein as well as with a needle means.

Needle biopsies are performed with devices of this type with aspiration of tissue and tissue fluid, a preferred field of application being the biopsy of the thyroid gland, but other organs, e.g., the prostate, the female breast, etc., are also considered for this diagnostic procedure. Depending on the thickness of the needle, distinction is frequently made between fine needle and large needle biopsy. Problems of diagnostic reliability that are associated with it are described, e.g., in the article “Large-Needle Aspiration Biopsy for the Preoperative Selection of Palpable Thyroid Nodules Diagnosed by Fine-Needle Aspiration as a Microfollicular Nodule or Suspected Cancer,” Angelo Carpi et al., American Journal of Clinical Pathology, 2000, 1.1.3, pp. 872-877.

The aspiration is carried out in prior-art devices either manually—by pulling out the plunger from the interior of the syringe housing—or with auxiliary mechanisms, e.g., a spring-actuated, releasable motion of the plunger.

Prior-art devices have a needle, which is arranged on a conventional syringe cylinder, a “syringe” for short, which can usually be fastened to the cone of the syringe cylinder with an attachment. To obtain cells, e.g., of the thyroid gland, the skin is cleaned and disinfected at the site of the puncture at the beginning of the procedure and the needle is then introduced into the thyroid tissue, with or without an additional anesthesia, and cells are brought into the needle channel or possibly into the interior of the syringe cylinder by suctioning, which is carried out by pulling the plunger in the syringe cylinder. Such puncturing may be performed blindly or under ultrasonic monitoring, and the cells obtained are then transferred into a vial for further diagnostic purposes, e.g., microscopic or chemical tests. Fine-needle biopsy of the thyroid gland is frequently preferred because it is nearly painless and has a low rate of complications only.

One drawback of the prior-art fine-needle biopsy is that only a single needle is used and material is therefore delivered from a defined location only. However, a tumor, which is possibly missed, may also be located in the immediate vicinity of healthy material. Fine-needle biopsy, which is not informative, may subsequently cause patients who are suspected of having a carcinoma (e.g., cold nodules of the thyroid gland) to be operated on, even though the probability of a carcinoma of the thyroid gland is between 5% and 15% in cold thyroid nodules. A liberal surgical strategy, i.e., surgery in all patients in whom carcinoma is suspected, without selection by fine-needle biopsy, must be called unethical because of the needless loss of the organ, high costs, etc.

Attempts are made for these reasons to increase the sensitivity of fine-needle biopsy by subjecting patients with thyroid nodules to puncturing several times in the course of a check-up, namely, in different regions of the thyroid gland. However, this strategy is not received well by patients and is therefore performed only rarely.

It shall be noted at this point that besides devices for needle biopsy, in which material is aspirated by means of a syringe cylinder and a needle, there also are puncturing devices that use a plurality of needles. For example, WO 01/52742 A1 discloses a device with a plurality of biopsy needles, namely, three biopsy needles in one exemplary embodiment. However, no aspiration is performed here, but the needles are only guided displaceably in channels, they cut out specimens from the tissue and can then be retracted.

A similar device with a plurality of needles, six needles in one exemplary embodiment, can be found in U.S. Pat. No. 5,415,182 A. Special biopsy needles are used here as well, which are formed by cannulae each and elongated stylets, which are displaceable therein, cut out specimens and can be retracted.

The said devices with a plurality of biopsy needles have a complicated design and are obviously not intended for use as disposable devices. Cleaning and sterilization seem to be utterly complicated.

It shall be mentioned that devices with a single needle means are also known, with which a plurality of specimens can be taken one after another. Such devices are shown, for example, in U.S. Pat. No. 6,083,150 or in EP 1,250,890 A2. In the case of these devices, which are likewise of a highly complicated design, specimens can be collected one after another in a device, but this does not eliminate the problem that a plurality of incisions and a corresponding stress of the patient are necessary. These prior-art devices are used above all for biopsies in hollow organs, e.g., the stomach and the intestines.

Hypodermic needle-like devices with a plurality of needles have become known as well. Thus, DE 1 907 296 A shows an adapter for hypodermic needles, which is used to connect a larger number of hypodermic needles, 7 needles in the example being shown, with a hypodermic needle and is to be used especially for subcutaneous injections of, e.g., local anesthetics. A similar device is described in DE 30 35 009 A1, in which the adapter can be screwed onto the outlet of a hypodermic needle, whereas it is attached by plugging according to the aforementioned document. This device also shall make possible the locally distributed penetration of a medicine, e.g., intramuscularly or subcutaneously. A suction device in the case of snakebites and insect bites according to DE 202 12 639 U1 likewise has a plurality of needles, which are seated in a mobile plunger, which is first pushed downward during the use of the device, while the needles penetrate into the skin. A second plunger is then pushed upward and it suctions fluid in terms of removing snake venom. Both plungers are moved automatically after release by spring force.

One problem of needle biopsy is that undesired tissue fluids and/or blood may also be aspirated during the pulling out of a needle after the puncturing operation proper. This problem is due to the vacuum that is still present in the syringe cylinder after puncturing. If a needle is pulled out of the patient's body after the aspiration of a target tissue, the tip of the needle is pulled, e.g., through vessels or other tissue parts, and fluid or tissue, whose examination is not desired or necessary, is aspirated.

One object of the present invention is to create a simple device, with which the above-mentioned drawbacks of fine-needle biopsy can be counteracted. In light of the increasing cost pressure in health care, the devices have a simple design and be correspondingly inexpensive.

This object is accomplished with a device of the type described in the introduction, in which the needle means has, according to the present invention, a plurality of puncture needles, whose channels open into the interior of the cylinder, and a ventilating means is provided for the interior of the syringe cylinder, by means of which the volume between the bottom and the plunger can be temporarily connected to the environment.

Thanks to the present invention, a number of tissue specimens can be taken simultaneously with a single application, and the stress for the patient is substantially reduced compared to the case in which an equal number of specimens are taken sequentially, and the risk of aspirating undesired matter, which is increased to a multiple in case of a plurality of needles, is reduced.

A highly advantageous embodiment of the device according to the present invention is characterized in that it has a stop means, which limits the depth of penetration of the needles into the body in a defined manner. At least one spacer, which has holes associated with the needles and can be pushed over the needles in order to limit the depth of penetration thereof into the body, may be provided as a stop means in one variant. The desired depth of penetration can be set in this manner, which is performed, for example, after a preceding ultrasound examination.

Provisions are made in another advantageous embodiment for a specimen container to be associated with each needle and for the specimen containers to be combined into one unit, which can be temporarily connected with the plurality of needles to empty the collected specimens into the containers. All specimens can thus be transferred into the specimen containers by a single motion of the plunger. It is advantageous in this case if the specimen container unit can be attached to the syringe cylinder by plugging, a groove-and-web arrangement on the specimen container and on the cylinder making it possible to noninterchangeably assign the individual needles to the specimen containers. If such a variant of the device is used, the assignment of the specimens taken to individual areas of the examined organ is ensured.

Furthermore, it is advantageous if a closing means is associated with the specimen containers, wherein the said closing means may have a closure for every individual specimen container and the closures for the specimen containers can be captively connected with these said specimen containers. Such a closing means prevents contamination of the specimens without the loss thereof already immediately after the puncture.

It is advantageous in terms of protecting the needles, on the one hand, and the staff, on the other hand, if a common protective sleeve, which can be attached by plugging over the needles and the syringe cylinder, is provided for all needles.

It is usually desirable for the specimens taken to fill only the cannulae of the needles, and it is undesirable that the specimens enter into the interior of the syringe cylinder. Provisions are made for this reason in one embodiment for arranging a filter means in the path between the opening of the channels into the tips of the needles and the interior of the syringe cylinder. The filter means may advantageously comprise individual filter inserts in the tip-side end area of the needles.

An advantageous variant is characterized in that the ventilating means is formed by at least one overflow channel, which is formed at a spaced location from the bottom of the syringe in the inner wall of the cylinder, the length of the channel in the direction of the axis of the cylinder making it possible to temporarily connect the volume between the bottom and the plunger with the interior of the cylinder located above the plunger via the at least one overflow channel. This ventilating means makes it possible to let out the vacuum and thus to allow blood and other undesired tissue parts to flow in during the pulling out of the needles.

The ventilating means may advantageously be formed by a vent hole, which passes through the wall of the cylinder and is located at a spaced location from the bottom of the said cylinder.

Provisions are made in another simple variant for a vent hole, which is closed by means of a closing piece during use, to be provided in the cylinder, wherein this can, however, be actuated manually in the sense of a temporary release of the hole.

Another possible and simple embodiment is characterized in that a vent hole, which is closed during use but can be opened in the sense of a temporary release of the hole, is provided in the plunger.

It is expedient for the simple but reliable setting of the plunger position after the aspiration of a desired volume if at least one indicator projection, which projects from the inner wall of the cylinder and can be overcome by the plunger, is provided at a spaced location from the bottom of the cylinder.

To make possible an easier penetration and/or to obtain specimens located at different depths, it may be advisable for at least some of the puncture needles of the needle means to have different lengths.

The present invention as well as other advantages will be explained in greater detail below on the basis of exemplary embodiments, which are illustrated in the drawings. In the drawings,

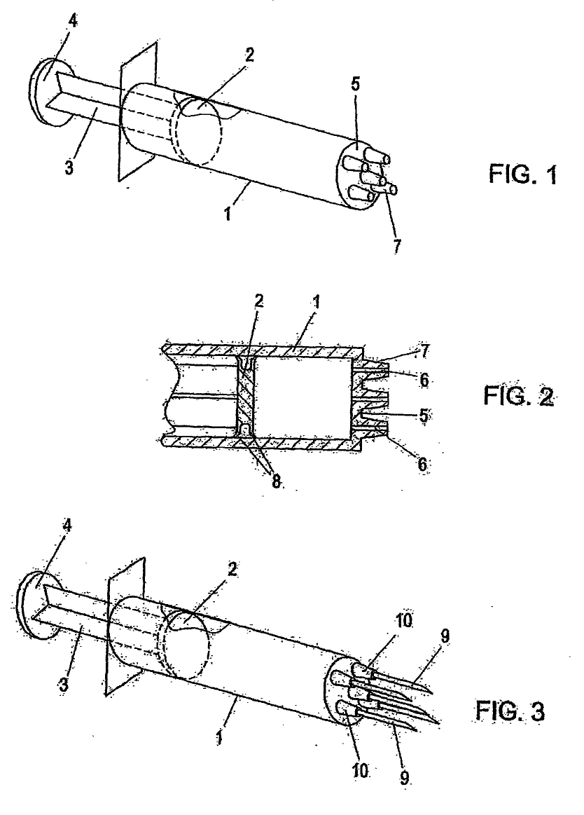

FIG. 1 shows a schematic perspective view of a device according to the present invention with five attachments for needles,

FIG. 2 shows a section through part of the device according to FIG. 1,

FIG. 3 shows the device with the needles attached in a view similar to that in FIG. 1,

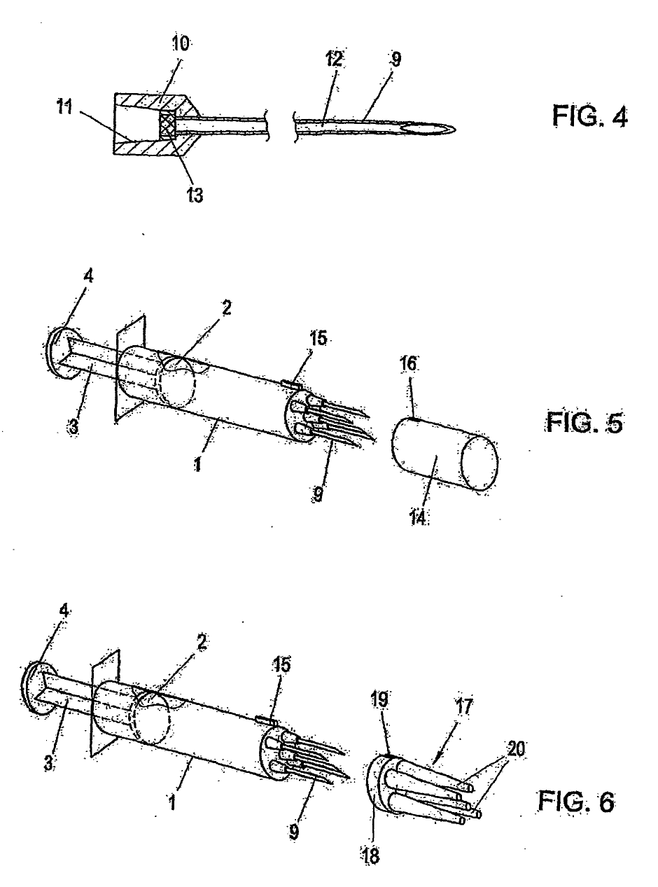

FIG. 4 shows a needle with a filter in a schematic sectional view,

FIG. 5 shows, likewise in a perspective view, another embodiment of the device according to the present invention with an attachable protective sleeve,

FIG. 6 shows an embodiment of the device according to the present invention with an attachable and removable specimen container,

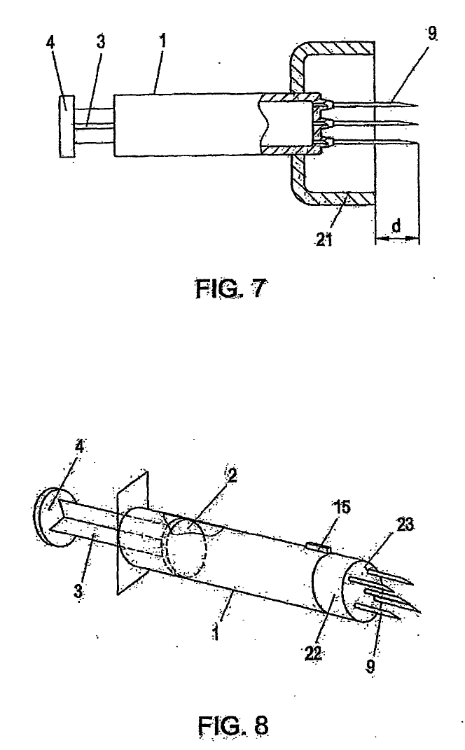

FIG. 7 shows a partially cut-away side view of an embodiment of the present invention with a stop means for limiting the depth of penetration of the needles,

FIG. 8 shows a schematic diagram of another embodiment with a spacer that can be pushed over to limit the depth of penetration,

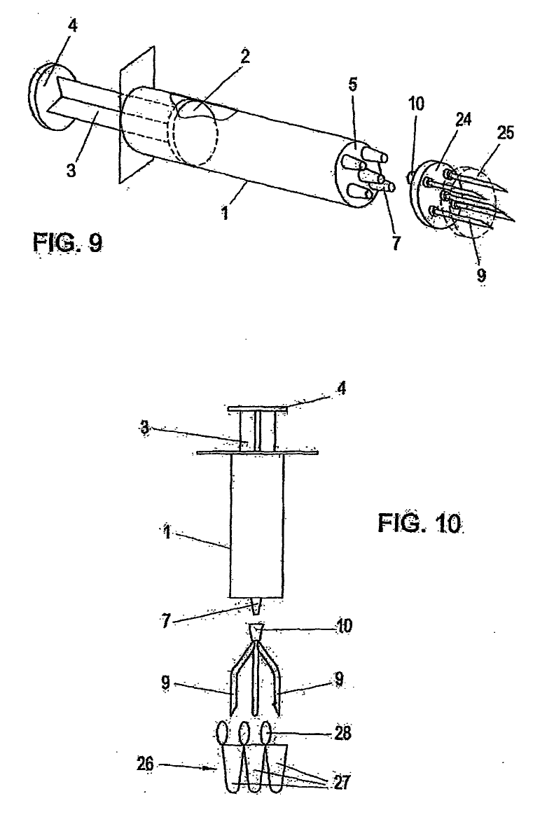

FIG. 9 shows a schematic diagram of an embodiment of the present invention with a holder for the needles,

FIG. 10 shows a schematic side view of an embodiment of the present invention, in which a plurality of needles are connected with a suction syringe via a single, common attachment,

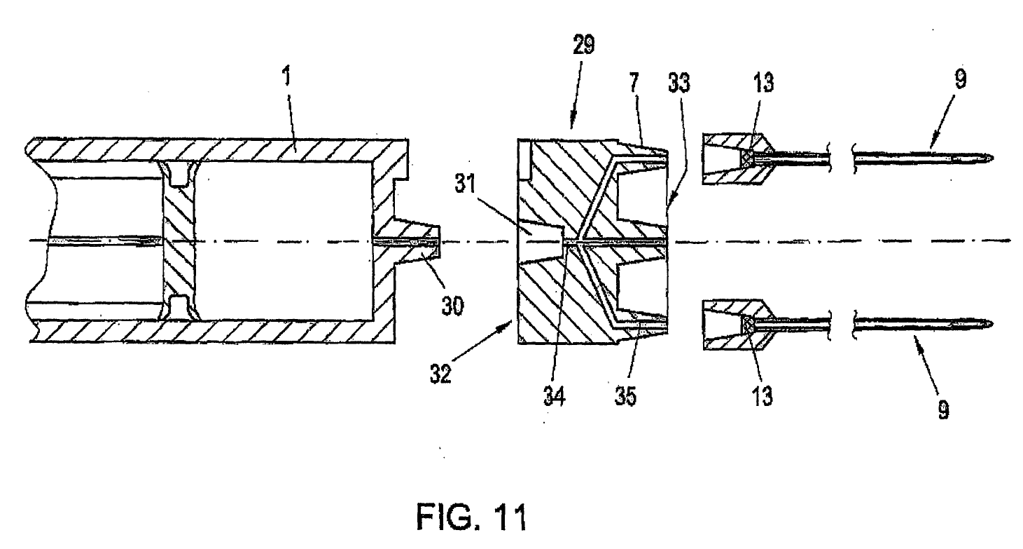

FIG. 11 shows a schematic side view of another variant of the present invention, in which a plurality of needles are connected with a suction syringe via an adapter,

FIG. 12 shows a variant of the present invention with a ventilating means designed as an overflow channel,

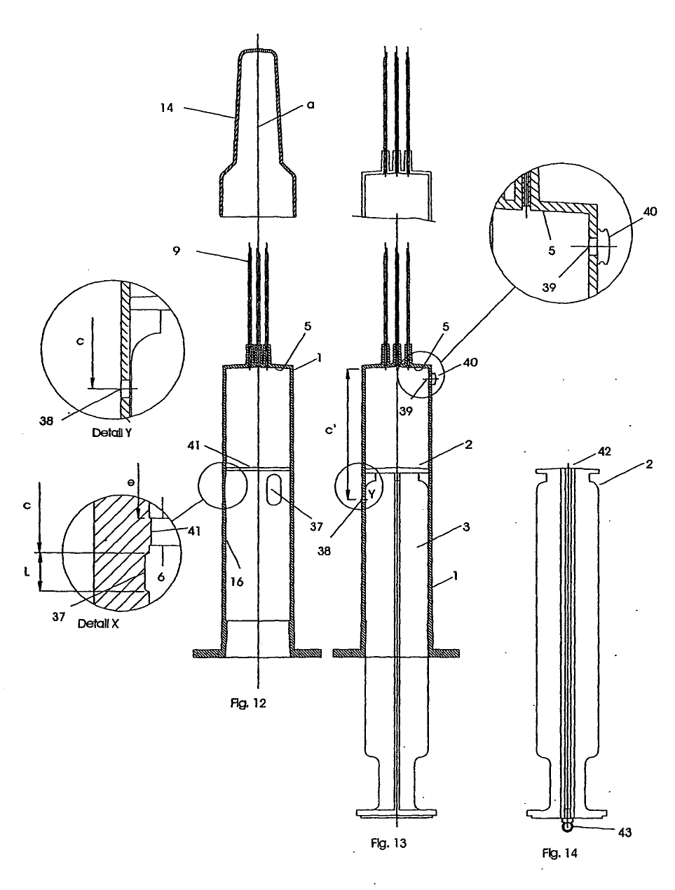

FIG. 13 shows a variant in which two ventilation possibilities with holes in the cylinder wall are illustrated, and

FIG. 14 shows an embodiment of a plunger with a central, closable ventilating channel.

As is apparent from FIG. 1 and FIG. 2, a device for needle biopsy according to the present invention has a syringe cylinder 1, in which a plunger 2 is displaceable. The plunger 2 is connected in this case with a handle 4 via a shaft 3 in the known manner, so that the plunger can be pulled out of the cylinder 1 in the sense of aspiration, i.e., suctioning, and can be pushed into [the said cylinder] in the sense of ejection.

A plurality of outlets 6, five outlets 6 in this case, which also pass through a cone 7 each, are provided in the bottom 5 of the syringe cylinder 1. The syringe cylinder 1, its bottom 5 and the cones 7 may be injection-molded from a plastic in one piece. Likewise, the plunger 2, the shaft 3 and the handle 4 may consist of a plastic. The plunger 2 may optionally consist, at least partly, of a soft plastic and have one or more circular sealing lips 8 in the known manner.

FIG. 3 shows the device according to the present invention with five attached puncture needles 9, such a needle being shown in FIG. 4. Each of the needles 9 has an attachment 10, which has an inner cone 11 matching the cones 7. The attachment 10 may consist of a plastic and molded in one piece with the needle 9 consisting of steel. The needle 9 itself is advantageously designed as a so-called “atraumatic” needle, i.e., as a needle provided with a tip with a special bevel.

A filter means may be arranged in the path between the opening of the needle channel 12 into the tip of the needle and the interior of the syringe cylinder 1 in order to prevent the aspiration of specimen material into the interior of the syringe. A filter insert 13 is arranged at the site of opening into the inner cone 11 at the inner end of the needle 1 in this exemplary embodiment.

A common cylindrical protective sleeve 14, which can be attached to the syringe cylinder 1 by plugging and then covers all needles 9, is provided for all needles 9 in the embodiment shown in FIG. 5. A short, outwardly projecting web 15, which engages a groove 16 or a slot of the cover when the protective sleeve 14 is attached by plugging, is arranged at the lower end of the syringe cylinder 1. This can improve the seating of the protective sleeve 14. The special significance of such a web-and-groove connection is explained in connection with a variant that will be described below.

FIG. 6 shows a specimen container unit 17, which belongs to the device according to the present invention and has an annular collar 18, with which it can be attached to the bottom-side end of the syringe cylinder 1. A groove 19 in the collar 18 cooperates with the web 15 of the syringe cylinder 1 when the specimen container unit 17 is attached, so that there is an unambiguous association between each of the needles 9 and individual specimen containers 20 of the unit 17. The web 15 may be used, furthermore, to set the position of the device during the biopsy procedure, e.g., it may be directed upwards or downwards. A preservative fluid may be present in the specimen containers 20. The entire specimen container unit 17 and/or every individual container 20 can be closed by means of a closure, which is not shown here.

Since it is often desirable, e.g., on the basis of a preceding imaging examination, to set the depth of penetration of the needles 9, a stop means, which comes into contact with the skin after the needles have penetrated into the body by a defined amount and prevents the further penetration of the needles, is also provided within the framework of the present invention. FIG. 7 schematically shows a stop ring 21, which surrounds the needles 9 from the outside and can be screwed, for example, on the syringe cylinder 1 on the outside, as a result of which the depth of penetration designated by d in FIG. 6 can also be set.

Another embodiment of a stop means is shown in FIG. 8. A spacer 22, which has a cylindrical design and has holes 23 associated with the needles 9, can be pushed over the needles 9 in the manner shown until it comes into contact with the bottom of the syringe cylinder 1 on the outside. The depth of penetration of the needles 9 can be set as desired with a set of spacers of different thicknesses.

FIG. 9 shows a holder 24, which has a disk-shaped design and sets the positions of the needles 9 in relation to one another. This holder 24 may consist of a plastic and be manufactured in one piece with the attachments 10, which likewise consist of a plastic. As an alternative or in addition, such a holder may also be arranged in the area of the metallic needle shafts 9, which is indicated by a holder 25 indicated by broken lines in FIG. 9.

In the embodiments explained so far, each needle can be connected individually with the syringe cylinder 1, which is performed via individual attachments 10 and cones 7. However, a plurality of needles or needle shafts may also be in connection, as an alternative, with a single attachment, which is shown in FIG. 10. The syringe cylinder 1 has a single cone 7 here. Five puncture needles 9, of which only three are visible because of the lateral view selected, are arranged in a fork-shaped pattern and open into a single attachment 10, which can be detachably connected with the cone 7 of the syringe cylinder 1. In the range of their depth of penetration into tissues, the shafts of the needles 8 advantageously extend in parallel to one another, just as in the above embodiments.

FIG. 10 shows yet another specimen container unit 26, in which the individual specimen containers 27 have a closure 28 each, which can be folded away individually and is captively connected with the container.

The variant of the present invention that is shown in FIG. 11 has an essentially cylindrical adapter 29, which is used to connect the syringe cylinder 1 with a plurality of puncture needles 9, for example, five puncture needles 9. Contrary to the embodiments described before, the syringe cylinder 1 has only a single cone 30 here, which can cooperate with an inner cone 31, which lies in a face 32 of the adapter 29.

A plurality, for example, five cones 7, which can cooperate with the needles 9 or, more precisely with the attachments 10 thereof, are provided on the opposite face 33 of the adapter 29.

The hole of the cone 30 is in fluid-conducting connection with the holes of the cones 7 via channels in the interior of the adapter 29, in this case via a central channel 34 and from this via branch channels 35 leading to the five cones 7.

To use the device according to FIG. 11, the adapter 29 is attached, on the one hand, by plugging to the syringe cylinder 1, when the cone 30 sealingly cooperates with the inner cone 31. The face 32 facing the syringe cylinder 1 is designed such that it can be in contact with the bottom 5 of the syringe cylinder 1 when the adapter 29 is attached by plugging to the syringe cylinder 1. This guarantees the required stability during the insertion of the needles 9. It is also possible as an alternative to support the adapter 29 by a “punctiform” contact with the syringe cylinder 1. In addition, a means securing against rotation, for example, in the form of a web 36, which projects at the syringe cylinder 1 and engages a groove 37 formed in the face 32 of the adapter 29 when the adapter 29 is attached, similarly to the web-and-groove connection 15-19 shown in FIG. 6, may be provided between the syringe cylinder 1 and the adapter 29.

A filter means 13 can prevent puncture material from penetrating into the interior of the cylinder in this embodiment as well. As is shown, this filter means 13 can be seated in the individual needles 9 in the form of filter inserts 13. As an alternative, a single filter insert may also be seated in the central channel 34 of the adapter 29, which is preferably made of a plastic.

The embodiment according to FIG. 11 may, of course, also be used with a specimen container according to FIG. 9, in which case a web, similar to the web 15 in FIG. 6, can be provided for this on the adapter 29.

A device for needle biopsy according to the present invention may be advantageously designed as a disposable device, already packaged in a sterile manner for use. However, other embodiments are also possible, especially ones in which the aspiration takes place automatically, e.g., by spring force after release.

During the use of the device according to the present invention, the device with the needles 9 is placed on the skin—after the cleaning and disinfection of the skin and the optional use of a corresponding spacer (FIG. 7, 8)—and pushed in to the stop. The tissue specimens are now aspirated into the needles 9 either by pulling out the plunger 2 manually or by the above-mentioned automatic aspiration. After pulling the needles 9 out of the body, e.g., a specimen container unit 17 according to FIG. 6 is attached to the syringe cylinder, and the specimens are transferred by pushing down the plunger 2 into the individual specimen containers 20, which are then closed with a suitable closure and are available for the further study of the specimens.

Many other variants, which are not shown here, are possible within the framework of the present invention. The device according to the present invention may also have a reusable design, for example, like a prior-art multipipette system, which has a single handle and is used to simultaneously aspirate different specimens from pipettes. It is, of course, also possible to take tissue specimens from body parts opened surgically, e.g., during a surgical procedure, with a device according to the present invention.

Adaptation to the particular conditions is possible by selecting the needle length. It is also possible to use needles of different lengths within one set of needles, which leads to two different effects:

On the one hand, specimens located at different depths can be taken as a result, and, on the other hand, the force needed for stabbing is reduced, because not all needles need to penetrate simultaneously into the skin or tissue.

If one or more needles is/are not to be used for a puncture procedure, provisions may be made to attach suitable closures—instead of the needles—to the corresponding cones of the syringe cylinder by plugging.

It shall also be mentioned that the needles may be nondetachably connected with the syringe cylinder, so that no further preparatory actions are needed after a corresponding disposable device has been removed from its sterile packaging.

Such an embodiment with needles 9 provided nondetachably can be seen in FIG. 12. The needles are seated next to each other at very closely spaced locations here, there being five needles 9 in this case, which can be covered by a protective sleeve 14 similar to that according to FIG. 5. Just as the embodiments according to FIGS. 13 and 14 below, the embodiment according to FIG. 12 has as a peculiarity a ventilating means, whose purpose and design shall be described below.

When a biopsy, for example, of the thyroid gland, is performed with a device corresponding to the present invention, the physician first pierces through the skin and other tissue layers, which usually contain many blood vessels, until the tips of the needles are located in the aspiration area proper. By pulling up the plunger, tissue is then aspirated into the interior of the syringe, and the needles are finally pulled out of the patient's body. It was found that the latter operation can cause problems insofar as vacuum usually still prevails in the interior of the cylinder after the tissue aspiration. As a result, undesired material, above all blood from tissues that are rich in blood, i.e., a material that is undesirable for the biopsy proper, is also aspirated in the course of the pulling out of the needles.

To avoid this drawback, a ventilating means is provided for the interior of the syringe cylinder 1, by means of which the volume between the bottom 5 and the plunger 2 can be temporarily connected with the environment.

According to FIG. 12 with its “detail X,” this ventilating means is formed by a plurality of, e.g., three overflow channels 37, which are formed in the inner wall 1i of the cylinder 1 at a distance c from the syringe bottom 5. The length l of the channels 37 in the direction of the axis of the cylinder is selected to be such that the volume between the bottom 5 and the plunger 2 is connected with the interior of the cylinder that is located above the plunger when the tightest, here disk-shaped section of the plunger is moving over the channels 37 or is located in the area of the said channels. At a short distance in front of the channels 37, at least one indicator projection 41, which, though being able to be overcome during the pulling out of the plunger 2, does perceptibly indicate to the physician that the desired aspiration volume has now been reached and the desired ventilation is taking place via the channels 37 during the further pulling out of the plunger 2, may project inwardly from the inner wall of the cylinder. As soon as this has occurred, the physician can pull the device out of the patient's body, i.e., leave the biopsy area with the needle tips, and be certain that undesired material, especially blood, will not be subsequently additionally aspirated.

It is obvious that the number of overflow channels 37 can be selected as desired; a single such channel or groove may be present as well.

Instead of an indicator projection 41, it is also possible to provide on the cylinder wall only a mark, which indicates the position of the plunger 2 or the desired volume.

FIG. 13 with its “details Y and Z” show another two possible embodiments of the ventilating means, in which case the ventilation takes place to the outside directly through the wall of the cylinder 1. According to detail Y, one vent hole 38 passes through the wall of the cylinder 1 at a distance c′ from the bottom 5. Automatic ventilation of the aspiration volume takes place in this case as well as soon as the plunger 2 is pulled over the vent hole 38.

According to “detail Z,” a vent hole 39 is closed by means of a closing piece 40, e.g., a small plug. This closing piece can be removed for ventilation. Two types of vent holes 38 and 39 are illustrated in FIG. 13, but only one variety will be present in practice.

Finally, FIG. 14 shows an embodiment in which the plunger 2 has an axially continuous vent hole 42, which is closed by means of a closure 43, e.g., a small plug, in the area if the plunger handle. The aspiration volume can be ventilated by opening the closure 43 after the aspiration of the material to be examined in this case as well, so that the undesired aspiration of blood, cerebrospinal fluid or the like during the pulling out of the needles from the patient's body can be avoided.

It shall be clear that the ventilation as described above may advantageously also be used in biopsy devices of this type that have only a single puncture needle.

We claim:

1. (canceled)

2. The device for needle biopsy in accordance with claim 18, wherein the device has a said stop means, which limits the depth of penetration of the needles into the body in a defined manner.

3. The device for needle biopsy in accordance with claim 2, wherein a spacer, which has holes associated with the needles and can be pushed over the needles in order to limit the depth of penetration into the body, is provided at least as a stop means.

4. (canceled)

5. (canceled)

6. (canceled)

7. (canceled)

8. (canceled)

9. The device for needle biopsy in accordance with claim 18, wherein a common protective sleeve, which can be attached by plugging to the syringe cylinder over the needles, is provided for all needles.

10. The device for needle biopsy in accordance with claim 18, wherein a filter means is arranged in the path between the opening of the channels into the tips of the needles and the interior of the syringe cylinder.

11. The device for needle biopsy in accordance with claim 10, wherein the filter means comprises individual filter inserts in the tip-side end area of the said needles.

12. (canceled)

13. (canceled)

14. (canceled)

15. (canceled)

16. (canceled)

17. (canceled)

18. A device for needle biopsy with a syringe cylinder, with a plunger displaceable therein as well as with a needle means,
wherein
the needle means has a plurality of puncture needles, whose channels open into the interior of the cylinder, and a ventilation means is formed by at least one overflow channel, which is formed at a distance from the syringe bottom in the inner wall of the cylinder, wherein the length of the channel in the direction of the cylinder axis makes it possible that the volume between the bottom and the plunger can be temporarily connected with the interior of the cylinder that is located above the plunger via at least one overflow channel.

19. The device in accordance with claim 18, wherein the needle means has a plurality of puncture needles.

20. The device in accordance with claim 18, wherein at least one indicator projection, which projects from the inner wall of the cylinder and can be overcome by the plunger, is provided at a distance from the bottom of the cylinder.

21. The device in accordance with claim 18, wherein at least some of the puncture needles of the needle means have different lengths.
